(19) United States
(12) Patent Application Publication
(10) Pub. No.: US 2013/0330309 A1
(43) Pub. Date: Dec. 12, 2013

(54) NUTRACEUTICAL AND PHARMACEUTICAL COMPOSITION

(71) Applicant: WHEY FORWARD HEALTH INDUSTRIES LTD, LONDON (GB)

(72) Inventor: JOSEPH PAUL FAULKNER-EDWARDS, CAMBRIDGE (GB)

(21) Appl. No.: 13/913,397

(22) Filed: Jun. 8, 2013

(30) Foreign Application Priority Data
Jun. 8, 2012 (GB) 1210100.2

(51) Int. Cl. A61K 38/16; A61K 47/42; A61K 47/22; A61K 31/198; A61K 47/02

(52) U.S. Cl. CPC A61K 38/168; A61K 31/198; A61K 47/02; A61K 47/22; A61K 47/42
USPC 424/93.51; 514/5.6; 424/94.64

(57) ABSTRACT

According to a first aspect, a composition comprises selenium, vitamin C, and at least 90% by weight of native or undenatured whey protein. According to a second aspect, a composition consists of cystine, selenium and vitamin C. According to further aspects, cosmetic formulations, pharmaceutical compositions and neutraceutical powders and beverages are provided, containing the compositions described above.

### CROSS-REFERENCE TO A RELATED APPLICATION

This application claims priority to United Kingdom Application No. 1210100.2, filed Jun. 8, 2012, which is incorporated herein by reference in its entirety.

### FIELD OF THE INVENTION

This invention relates to nutraceutical and pharmaceutical compositions.

### BACKGROUND OF THE INVENTION

Glutathione is not an essential nutrient, and can be synthesised in the body. It is a tripeptide, formed from the amino acids cysteine, glutamic acid and glycine. Cysteine is generally accepted to be the rate-limiting component in cellular glutathione synthesis, since this amino acid is relatively rare in foodstuffs.

Glutathione has multiple cellular functions. It is an antioxidant, which neutralises free radicals and reactive oxygen compounds. Free radicals and reactive oxygen species have been linked to the ageing process. Oxidative stress has also been linked to cancer, AIDS and neurodegenerative diseases.

Glutathione is essential in the regulation of nitric oxide, which is critical for life, and it is also used in metabolic and biochemical reactions such as DNA synthesis and repair. It also has a vital function in iron metabolism.

Cysteine, which is a component of glutathione, is an amino acid, which can be synthesised in humans. Cystine is a dimeric amino acid, formed by the dimerization of two cysteine residues, which covalently link to form a disulfide bond. Cystine is broken down intracellularly to form cysteine.

Whey protein is a naturally-occurring and abundant source of the three amino acids in glutathione. Whey supplements have been linked to the promotion of glutathione production, see for example “The influence of dietary whey protein on tissue glutathione and the diseases of aging”, Clin Invet Med. 1989 December; 12(6):343-9.

Whey protein can be processed in three ways to yield whey isolate, whey concentrate or whey hydrolysate. The three different whey protein forms result from different processes that are used to filter the proteins. Whey protein isolates yield a higher percentage of pure protein, and can be filtered enough to be virtually lactose, carbohydrate, fat and cholesterol free. Whey protein isolates are at least 90% protein by weight.

Undenatured whey proteins have usually been cold-processed, in order to avoid denaturing the proteins contained in the whey. Undenatured whey proteins are high in cystine, which is beneficial for raising glutathione levels.

In some countries, whey products are now being recognised for not only their high nutritional value, but also their capability for improving health and preventing disease. For example, whey products have been proposed as an anti-cancer treatment.

Whey has also been associated with glutathione synthetics. Vitamin C (ascorbic acid), lactoferrin and selenium have each been linked to glutathione production, but not conclusively. For example, see “Vitamin C elevates red blood cell glutathione in healthy adults”, Am J Clin Nutr. 1993 July; 58(1):103-5, “Lactoferrin-Protector against Oxidative Stress and Regulator of Glycolysis in Human Erythrocytes”, Z. Naturforsch. 58c, 256-262 (2003), “Effect of selenium-saturated bovine lactoferrin (Se-bLF) on antioxidant enzyme activities in human gut epithelial cells under oxidative stress”, Anticancer Agents Med Chem. 2011 October; 11(8):762-71 and “In vivo and in vitro influence of selenium on DNA/RNA synthesis in spleen and lymphocytes in culture-possible mediation of changes in GSH/GSSG ratio”, Indian J. Exp Biol. 2001 January; 39(1):25-8.

Some of these agents have been formulated with each other (but not all of them), but the heath benefits of such formulations have not been extensively researched.

GB2458466 describes compositions for hair, skin and nail health maintenance. It comprises many different ingredients, including cystine, selenium and vitamin C (ascorbic acid), in relatively low amounts. WO2004/060487 discloses a similar composition, for the treatment of HIV or AIDS.

WO2008/001086 describes a nutritional formulation for the treatment of Crohn's disease. It contains whey protein (6 g/100 g powder), vitamin C and selenium. The large proportion of the formulation (58 g/100 g powder) is maltodextrin. Other components are also present in the formulation.

### BRIEF SUMMARY OF THE INVENTION

It has surprisingly been found that native or undenatured whey protein, which is an effective source of cystine, is not only much more effective than cysteine at promoting glutathione production, but it works synergistically with selenium and vitamin C to form a composition that has much improved health benefits, when compared to traditional whey protein formulations, or vitamin C or selenium alone. Without wishing to be bound by theory, it is believed that the components in the native or undenatured whey protein (which should be the main component of the formulation), work synergistically with the selenium and vitamin C, to form a composition with much improved health benefits.

Therefore, according to a first aspect of the invention, a composition comprises selenium, vitamin C, and at least 90% by weight of native or undenatured whey protein

It is also believed, therefore, that cystine, vitamin C and selenium will work synergistically.

Therefore, according to a second aspect, a composition consists, or consists essentially, of cystine, selenium and vitamin C.

According to further aspects, cosmetic formulations, pharmaceutical compositions and neutraceutical powders and beverages are provided.

### DETAILED DESCRIPTION OF THE INVENTION

### Description of Preferred Embodiments

According to a first aspect of the invention, a composition comprises selenium, vitamin C, and at least 90% by weight of native or undenatured whey protein. It is believed to be necessary to have the whey protein present in a large amount, in order to promote glutathione production, and achieve the synergy with the vitamin C and the selenium.

In another aspect, a composition of the subject invention contains cystine. It is also believed that cystine, vitamin C and selenium work synergistically. Therefore, according to one aspect, a composition of the subject invention consists, or consists essentially, of cystine, selenium and vitamin C.

The term “consisting essentially of,” as used herein, limits the scope of the ingredients and steps to the specified materials or steps and those that do not materially affect the basic and novel characteristic(s) of the present invention, e.g., compositions and methods for improving health by promoting glutathione production. For instance, by using “consisting essentially of,” the therapeutic composition does not contain any unspecified ingredients that have a direct beneficial or adverse therapeutic effect on health via the role of glutathione. Also, by using the term “consisting essentially of,” the compositing may comprise substances that do not have therapeutic effects attributable to an increase in glutathione production; such ingredients include carriers, excipients, adjuvants, flavoring agents, etc. that do not affect glutathione production.

In such as embodiment, the source of cystine in a composition of the invention may be lactoferrin, undenatured whey protein isolate (WPI) or native whey protein (from milk), which is preferably cold-processed, or a combination thereof. Alternatively, isolated cystine may be present in a composition of the invention. Isolated cystine is commercially available. For example, it can be isolated from human hair. A composition of the invention preferably contains one or more of these sources. For example, it may contain a combination of two or three of these cystine sources.

Preferably, the native or undenatured whey protein is whey protein isolate. Preferably, the whey protein is both native and undenatured. The whey protein may be from whey protein concentrate. More preferably, it is WPI 90. Undenatured and native whey protein is preferred as it is very effective at promoting glutathione production.

Native Whey Protein is obtained through a very specific process that differs from standard whey protein: the proteins are extracted directly from milk using membrane technologies at low temperature (microfiltration and ultrafiltration), leaving only biologically active whey protein.

Native Whey Protein is only pasteurised once avoiding unnecessary secondary heat treatments that can denature cystine rich peptides irreversibly. It is not a byproduct of cheese, and therefore not denatured. It is also very high in cystine, and is therefore preferred in the present invention.

Any selenium-containing ingredient may be used as the selenium source in the invention. In a preferred embodiment, a composition of the invention comprises selenium yeast, as the selenium source. Commercial formulations contain varying amounts of selenium, but in a preferred embodiment, the selenium yeast used in the invention contains between 1000 mcg/g and 3000 mcg/g of selenium. More preferably, the selenium yeast used in the invention contains 2000 mcg/g of selenium. Selenium is an essential component of two important antioxidant enzymes, glutathione peroxidise, which recycles glutathione; and thioredoxin, which recycles vitamin C.

Vitamin C is also present in compositions of the invention. Any form of vitamin C is suitable for use in the invention. Ascorbic acid is one form of Vitamin C that can be used in the invention. The term ascorbic acid also includes salts thereof, i.e. ascorbates. For example, calcium ascorbate is suitable for use in the invention. Without wishing to be bound by theory, vitamin C promotes red blood cell glutathione levels, which results in an increased antioxidant capacity of the blood. This is vital for immune function and disease protection.

Preferably, a composition of the invention comprises (and preferably consists of) whey protein isolate, selenium yeast and vitamin C. Preferably, a composition of the invention also contains additional lactoferrin.

Preferably, a composition of the invention comprises:

selenium yeast;

vitamin C;

optionally lactoferrin; and

at least 90% by weight native or undenatured whey protein

More preferably, a composition of the invention comprises:

0.05-0.5% by weight selenium yeast;

1-5% by weight vitamin C;

optionally (but preferably) 0.1-3% by weight lactoferrin; and

at least 90% by weight whey protein isolate.

In a preferred embodiment, a composition of the invention consists of:

0.05-0.5% by weight selenium yeast;

1-5% by weight vitamin C;

Up to 3% by weight lactoferrin; and

a remainder of native or undenatured whey protein.

A preferred amount of selenium yeast (which is preferably 2000 mcg/g) is 0.1 to 0.2% by weight. For the avoidance of doubt, as used herein, % by weight means % by weight of the total dry powder composition.

A preferred amount of vitamin C is 2-3% by weight.

Lactoferrin is not necessarily present in the composition, since its function is to increase the amount of cystine present in the formulation;however, in a preferred embodiment, it is present. In a more preferred embodiment, it is present in an amount of up to 1% by weight, more preferably, from 0.25 to 1% by weight.

A composition of the invention is preferably formulated as a dry powder. The dry powder may be formulated into a tablet, for example, for use as a pharmaceutical composition. Alternatively, it may be reconstituted with a liquid, for example, water, to be consumed as a beverage.

A composition of the invention may be useful in therapy of a subject, e.g as a pharmaceutical composition or as a cosmetic formulation. For therapeutic use, a composition according to the invention may slow the ageing process, optimise immune function, and/or be useful as an antiviral, antimicrobial, or anticancer agent.

The term “subject,” as used herein, describes an organism, including mammals such as primates, to which the compositions according to the present invention can be provided. Mammalian species that can benefit from these compositions include, but are not limited to, apes, chimpanzees, orangutans, humans, monkeys; and other animals such as dogs, cats, horses, cattle, pigs, sheep, goats, chickens, mice, rats, guinea pigs, and hamsters.

A composition of the invention may also be useful as a nutraceutical.

Preferably, a cosmetic formulation comprises a composition according to the inventionthat may be used to slow the ageing process.

A composition of the invention may be formulated and packaged as a ready-to-use-drink beverage. In certain embodiments, the beverage composition may be pre-mixed with, or dissolved in a liquid such as water, preferably spring water. In specific embodiments, the ready-to-drink beverage comprises about 80-99 weight percent (wt %) of liquid of the total weight of the beverage. In further embodiments, the beverage composition can be packaged as an edible composition or concentrate, such as a dry mix (e.g., powder) or a liquid concentrate for later reconstitution with one or more liquids to form a beverage. The concentrated composition may be associated with instructions for preparing the beverage composition. In another embodiment, a beverage concentrate may be packaged as a gel, sachet, capsule, or tablet that is consumed with liquid. When provided in these forms, the beverage composition may comprise instructions to mix or consume with an amount of liquid that is equal to about 80-99 wt % of the prepared beverage composition.

Dry powder can also be added to foodstuffs (providing that it is not cooked or heated), e.g. for use in health bars and the like.

In another embodiment, a composition of the invention is formulated as a shot or pouch. A shot is a small, unit dose sealed presentation, preferably comprising between about 5 and about 500 ml or between about 50 and about 200 ml of the beverage composition of the invention. A pouch presentation may contain between 50 and 500 ml of the beverage composition of the invention.

A beverage in accordance with the invention preferably comprises at least water or milk, whey protein isolate, selenium and vitamin C. Exemplary flavourings that may be suitable for at least certain formulations in accordance with this disclosure include citrus flavouring, spice flavourings and others. Preservatives can be added if desired, depending upon the other ingredients, production technique, desired shelf-life, etc. Additional and alternative suitable ingredients will be recognized by those skilled in the art given the benefit of this disclosure.

A beverage of the invention may be a clear solution, but can be coloured. It may also be translucent/cloudy, for example, if it comprises milk.

A beverage of the present invention may also include a clouding agent at a concentration range of from about 0 to about 100 ppm of clouding agent. Examples of clouding agents include, but are not limited to, ester gum, SAIB, starch components and mixtures thereof, with ester gum as the preferred clouding agent at a concentration range of from about 10 to about 50 ppm and more preferably from about 15 to about 35 ppm.

The present invention also relates to a beverage concentrate used to prepare the beverage already described herein. As used herein, the term “beverage concentrate” refers to a concentrate that is either in liquid or gel form or in essentially dry mixture form. The essentially dry mixture is typically in the form of a powder, although it may also be in the form of a single-serving tablet, or any other convenient form. The concentrate is formulated to provide a final and complete beverage as already described herein when constituted or diluted with water or other liquid.

In further embodiments, the composition may include optional additives such as antioxidants, amino acids, caffeine, colouring agents (“colorants”, “colourings”), emulsifiers, flavour potentiators, food-grade acids, minerals, micronutrients, plant extracts, phytochemicals (“phytonutrients”), preservatives, salts including buffering salts, stabilizers, thickening agents, medicaments, vitamins, and a combination of the foregoing additives. Those of ordinary skill in the art will appreciate that certain additives may meet the definition or function according to more than one of the above-listed additive categories.

The beverage compositions can be packaged, ready-to-drink, and can be shelf-stable. Any type of beverage packaging can be used to package the beverage composition including glass bottles, plastic bottles and containers (e.g., polyethylene terephthalate or foil lined ethylene vinyl alcohol), metal cans (e.g., coated aluminium or steel), lined cardboard containers, and the like. Other beverage packaging material known to one of ordinary skill in the art can be used.

A composition of the invention may be provided in a kit but also comprising a container with means for mixing a composition of the invention (in powder form), with a liquid (for example, water), in order to foim a beverage. Such a container preferably contains a stirrer, preferably a plastic blade, for mixing the beverage.

A stirrer of the invention is preferably powered by a motor, which spins the stirrer at least 8,000 rpm, to ensure that all the dry powder is blended with the added liquid. Preferably, the blade is blunt, to avoid mechanically denaturing any proteins.

A composition of the invention may be useful in therapy. Therapy according to the invention may be conducted in generally known manner, depending on various factors, such as the sex, age or condition of the patient, and the existence or otherwise of one or more concomitant therapies.

A pharmaceutical composition of the invention may be formulated into any suitable form, for example, a tablet, dry powder, oil-in-water emulsion. It may also be formulated for any suitable use of administration, such as oral or parenteral.

In a preferred embodiment, a composition of the invention is useful in slowing the ageing process, to optimise immune function, to raise energy levels, for use as an anti-viral, an anti-microbial or an anti-cancer agent.

The present invention also provides a cosmetic formulation comprising a composition according to the invention, to slow the ageing process.

The following Example illustrates one embodiment of the invention.

### EXAMPLE

A dry powder was prepared to the following specification:

| Ingredient | mg/Serve | g/serve | % |
|---|---|---|---|
| Native, undenatured whey | 19472.500 | 19.4725 | 97.3625 |
| protein isolate | | | |
| Selenium Yeast 2000 mcg/g | 27.500 | 0.0275 | 0.1375 |
| Vit C (ascorbic acid) | 500.000 | 0.5000 | 2.5000 |

The powder was then mixed with water to form a nutraceutical beverage.

The nutraceutical beverage was tested on a number of people of varying age, fitness and occupation, and a sample of the results is shown below.

The beverage was tested by a personal trainer and an osteopenia sufferer (the stage before osteoporosis). An increase in alertness and energy was reported.

The beverage was tested by a singer. After consuming the beverage each day for 9 months, an overall improvement in energy levels was noted, especially mid-afternoon. A significant reduction in the usual coughs, sore throats and colds was also noted, suggesting that the beverage promotes strengthening of the immune system.

Another test subject reported, in addition to the energy benefits, benefits with respect to digestion.

Generally, the test subjects reported a benefit with respect to traditional whey protein and nutraceuticals claiming health benefits. This supports the hypothesis that the selenium and vitamin C, at least, are working synergistically with the components in the WPI.

Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate).

The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, or consisting essentially of that element, unless otherwise stated or clearly contradicted by context).

I claim:

1. A composition comprising selenium, vitamin C, and at least 90% by weight of native or undenatured whey protein.

2. The composition according to claim 1, additionally comprising lactoferrin.

3. The composition according to claim 1, wherein the whey protein is whey protein isolate.

4. The composition according to claim 1, which comprises, as the selenium source, selenium yeast.

5. The composition according to claim 4, wherein the selenium yeast contains between 1000 mcg/g and 3000 mcg/g of selenium.

6. The composition according to claim 1, comprising:
0.05-0.5% by weight selenium yeast;
1-5% by weight vitamin C;
optionally 0.1-3% by weight lactoferrin; and
at least 90% by weight native or undenatured whey protein.

7. The composition according to claim 1, which is formulated as a dry powder or liquid concentrate.

8. A composition consisting essentially of cystine, selenium and vitamin C.

9. The composition according to claim 8, which is formulated as a dry powder or liquid concentrate.

10. A nutraceutical comprising either a composition according to claim 1, or a composition consisting of cystine, selenium and vitamin C.

11. A nutraceutical beverage comprising either a composition according to claim 1 dissolved in a suitable liquid, or a composition consisting of cystine, selenium and vitamin C dissolved in a suitable liquid.

12. A pharmaceutical composition comprising either a composition according to claim 1, or a composition consisting of cystine, selenium and vitamin C.

13. A cosmetic formulation comprising either a composition according to claim 1, or a composition consisting of cystine, selenium and vitamin C.

14. A kit comprising either a composition according to claim 1, or a composition consisting of cystine, selenium and vitamin C, wherein said kit further comprises a container suitable for mixing the composition with a liquid, to form a beverage.

15. The kit according to claim 14, wherein the container contains a stirrer or blade, which can rotate at a speed of at least 8,000 rpm, to blend the composition with a liquid.

16. A therapeutic method for slowing the ageing process; improving immune function; raising energy levels; and/or providing anti-viral, anti-microbial, and/or anti-cancer activity, wherein said method comprises administering, to a subject in need of such therapy, either a composition of claim 1, or a composition consisting of cystine, selenium and vitamin C.

17. A method for increasing glutathione production in a subject who has been diagnosed with a condition that would be improved by increased glutathione production, wherein said method comprising administering to the subject either a composition of claim 1, or a composition consisting of cysteine, selenium, and vitamin C.

18. The composition, according to claim 8, that consists of cysteine, selenium, and vitamin C.
